(19) United States
(12) Patent Application Publication
(10) Pub. No.: US 2024/0374492 A1
(43) Pub. Date: Nov. 14, 2024

(54) A SUNSCREEN COMPOSITION COMPRISING AVOBENZONE AND A METHOD FOR PHOTOSTABILIZATION

(71) Applicant: ISP Investments LLC, Wilmington, DE (US)

(72) Inventors: Hani M. FARES, Somerset, NJ (US); Donald I. PRETTYPAUL, Englenwood, NJ (US); Drupesh PATEL, Lake Hiawatha, NJ (US)

(73) Assignee: ISP Investments LLC, Wilmington, NJ (US)

(21) Appl. No.: 18/293,129

(22) PCT Filed: Jul. 13, 2022
(86) PCT No.: PCT/US22/36989

§ 371 Date: Jan. 29, 2024

Related U.S. Application Data
(60) Provisional application No. 63/227,468, filed on Jul. 30, 2021.

(51) Int. Cl. A61K 8/37; A61Q 17/04

(52) U.S. Cl. CPC A61K 8/37; A61Q 17/04; A61K 2800/49; A61K 2800/52; A61K 2800/591

(57) ABSTRACT

The present application relates to a sunscreen composition comprising (i) about 0.01 wt. % to about 20 wt. % of avobenzone, (ii) about 0.01 wt. % to about 20 wt. % of benzyl o-toluate; and (iii) about 0.01 wt. % to about 95 wt. % of at least one additive; and a method for solubilizing and photostabilizing avobenzone in a sunscreen composition using an effective amount of benzyl o-toluate.

### FIELD OF THE INVENTION

The present application relates to a sunscreen composition, and more particularly, to a sunscreen composition comprising (i) avobenzone, (ii) benzyl o-toluate, and (iii) at least one additive. The present application further relates to a method for solubilizing and photostabilizing avobenzone in a sunscreen composition using an effective amount of benzyl o-toluate.

### BACKGROUND OF THE INVENTION

It is well-known that sunlight radiation ranging from 290 to 400 nm is detrimental to organic materials including human skin and particularly radiation with wavelengths between 290 and 320 nm (UV-B region) is responsible for the occurrence of erythema and sunburns whose severity depends on exposure length. Furthermore, it has been ascertained that radiation between 320 and 400 nm (UV-A region) which is responsible for skin tanning can cause alterations and important damages in skin, especially in the case of sensitive skin or in the case of continuous exposure to the radiation. It has been shown that UV-A radiation causes damage to elastin and collagen that leads to skin ageing, and additionally, it can also be the cause of a number of phototoxic and photo allergic reactions. Moreover, the harmful nature of UV-B can also be enhanced by the presence of UV-A.

Many commercial products, such as personal care compositions (e.g., sunscreen compositions) offer protection against such harmful effects of UV-A or UV-B radiations include functional organic compound(s) comprising UV-A and/or UV-B screening agents. These functional actives often require solubilization in the form of a solution, an emulsion, or a dispersion, and wherein the solubilization is in aqueous or non-aqueous form. For example, a sunscreen composition containing functional organic compound(s) such as avobenzone (Escalol® 517) and/or bis-ethylhexyloxyphenol methoxyphenyl triazine (Escalol™ S) as active UV-A/UV-B absorbing ingredient requires a solubilization agent to prevent crystallization and keep them stable in a composition. Often these functional actives also require stabilization against photodegradation in the compositions. Several such solubilizers are known in the domain includes ethyl benzoate or a C12-C15 alkyl benzoate, however, the former compound is a strong irritant, and the latter is only a mediocre solvent for avobenzone.

One of the biggest technological challenges for sunscreen compositions is to effectively solubilize and photostabilize the functional active agents which often are lipophilic in nature, and at the same time provide protection against harmful UV radiations. The present application provides an effective means of meeting the aforementioned unmet needs.

U.S. Patent Publication 2005/0,152,858 assigned to ISP Investments Inc. discloses a sunscreen composition comprising an active or functional organic sunscreen UV-A and/or UV-B absorbing compound selected from avobenzone and/or benzophenone-3 solubilized in a phenylethyl ester which is an aryl carboxylic ester of 2-phenylethyl alcohol.

PCT Publication 2003/039,510 assigned to Sol-Gel Technologies Ltd. discloses a pharmaceutical or cosmetic composition comprising at least one active ingredient in combination with an oil or a mixture of oils, wherein the oil or mixture of oils alone or in combination with the active ingredient have a specific gravity higher than the specific gravity of water. The active ingredient can be a sunscreen agent such as UV-A absorber or UV-B absorber.

U.S. Pat. Nos. 7,166,275 and 7,691,363 assigned to ISP Investments Inc. disclose compositions of an active or functional organic compound solubilized in a phenyl ethyl ester comprising of 2-phenylethyl benzoate, toluate, or phthalate. The active or functional organic compound can be a solid organic compound, e.g., a personal care, cosmetic, sunscreen, pharmaceutical, agricultural, or industrial compound. Preferred actives include active sunscreen ingredients, e.g., UV-A and/or UV-B sunscreens such as avobenzone and/or benzophenone-3.

U.S. Pat. No. 10,328,003 assigned to ISP Investments Inc. discloses a photo protective topical composition, comprising a functional active and a solubilizing compound preferably selected from selected from cyclohexyl 3-toluate and cyclohexyl 4-toluate. The functional active can be selected from a personal care active, cosmetic care active, pharmaceutical active or a nutraceutical active.

U.S. Pat. No. 7,785,573 assigned to ISP Investments Inc. discloses active or functional organic compounds solubilized by an ester of an aryl alcohol, e.g., phenethyl, benzyl or substituted benzyl alcohol, and an alkyl or cycloalkyl carboxylic acid, or by a carbonate of the aryl alcohol and an alkyl or cycloalkyl carbonic acid.

U.S. Pat. No. 7,364,721 assigned to L'Oreal discloses a cosmetic composition including an active or functional organic compound which is solubilized by a safe and effective organic solvent such as an aryl carboxylic ester of 2-phenylethyl alcohol, e.g., 2-phenylethyl benzoate, 2-phenylethyl toluate or di-2-phenylethyl phthalate.

U.S. Pat. No. 9,717,931 assigned to L'Oreal discloses a cosmetic or dermatological photo protective composition (A) comprises at least 1,3,5-triazine derivative (I) and a mixture (II) comprising an oil (III) having at least an amide motif in its structure and an arylalkyl benzoate derivative (IV).

U.S. Pat. No. 7,368,105 assigned to L'Oreal discloses photostabilization of at least a dibenzoylmethane derivative against UV radiation, comprises linking with at least one arylalkyl benzoate derivative and a bisresorcinyl triazine compound.

U.S. Pat. No. 7,364,720 assigned to L'Oreal discloses a topically applicable, photostable cosmetic/dermatological photoprotective composition, comprising an effective UV-photoprotecting amount of at least one dibenzoylmethane UV-screening agent, and, as a stabilizing admixture therefor, a thus effective amount of at least one arylalkyl benzoate compound and at least one amide-based oil, formulated into a topically applicable, cosmetically/dermatologically acceptable medium.

In view of the foregoing, still there is a need to develop a technique for effectively solubilizing and photostabilizing UV actives such as avobenzone and also to solubilize bis-ethylhexyloxyphenol methoxyphenyl triazine using an effective amount of a solubilizer in the sunscreen compositions wherein, the solubilizer exhibits good odor.

Accordingly, it is an object of this invention to provide a sunscreen composition including one or more active or functional organic compound(s) comprising UV-A and/or UV-B screening agents, which is effectively solubilized and photostabilized using a safe, organic solubilizer composition.

A further object herein is to solubilize and photostabilize at least 10 wt. %, or at least 20 wt. % of UV screening agents, based on total weight of the sunscreen composition with an effective amount of solubilizer composition wherein the solubilizer composition exhibits good odor.

The foregoing and other objects and features of the invention will be made apparent from the following description.

### SUMMARY OF THE INVENTION

The primary aspect of the present application is to provide a sunscreen composition comprising: (i) about 0.01 wt. % to about 20 wt. % of avobenzone, (ii) about 0.01 wt. % to about 20 wt. % of benzyl o-toluate, and (iii) about 0.01 wt. % to about 95 wt. % of at least one additive.

In another aspect, the present application further includes about 0.01 wt. % to about 25 wt. % of at least one UV screening agent and/or physical sun blocker selected from but not limited to the group consisting of octisalate, homosalate, octocrylene, benzopheneone-3, bis-ethylhexyloxyphenol methoxyphenyl triazine, titanium dioxide, zinc oxide, titanium dioxide (and) isopropyl titanium triisostearate, zinc oxide (and) isopropyl titanium triisostearate, zinc oxide (and) triethoxycaprylylsilane, and combinations thereof.

In another aspect, present application provides a sunscreen composition that can effectively solubilize and photostabilize avobenzone and solubilize bis-ethylhexyloxyphenol methoxyphenyl triazine using an effective amount of a solubilizer composition benzyl o-toluate wherein, the solubilizer composition exhibits good odor.

Another aspect of the present application is to provide a method for solubilizing and photostabilizing avobenzone in a sunscreen composition comprising avobenzone, the method comprising adding about 0.01 wt. % to about 20 wt. % of benzyl o-toluate to the composition to stabilize the avobenzone against photodegradation and to effectively solubilize the avobenzone.

In another aspect, the present application provides a method for protecting the skin against UV radiation comprising applying to the skin an effective amount of a sunscreen composition comprising: (i) about 0.01 wt. % to about 20 wt. % of avobenzone, (ii) about 0.01 wt. % to about 20 wt. % of benzyl o-toluate, and (iii) about 0.01 wt. % to about 95 wt. % of at least one additive.

### BRIEF DESCRIPTION OF THE FIGURES

Further embodiments of the present application can be understood with the appended figures.

FIG. 1 illustrates the effect of solvent benzyl o-toluate on photostability of avobenzone.

FIG. 2 illustrates the effect of solvent 2-phenylethyl o-toluate on photostability of avobenzone.

FIG. 3 illustrates the effect of solvents such as C12-C15 alkyl benzoate and phenethyl benzoate on photostability of avobenzone.

FIG. 4 illustrates the effect of solvents such as N,N-diethyl m-toluamide and propylene glycol dibenzoate on photostability of avobenzone.

FIG. 5 illustrates the effect of solvents such as 1,3-butylene glycol dibenzoate and cyclohexyl benzoate on photostability of avobenzone.

FIG. 6 illustrates the effect of solvents such as cyclohexyl o-toluate and benzyl 4-tert-butylbenzoate on photostability of avobenzone.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of.”

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “preferred,” “preferably,” and variants thereof, refer to embodiments of the invention that afford certain benefits, under certain circumstances. However, other embodiments are also preferred, under the same or other circumstances. Furthermore, the recitation of one or more preferred embodiments does not imply that other embodiments are not useful and is not intended to exclude other embodiments from the scope of the invention.

References herein to “one embodiment” or “one aspect” or “one version” or “one objective” of the application include one or more such embodiment, aspect, version, or objective, unless the context clearly dictates otherwise.

The term “alkyl” refers to a functionalized or unfunctionalized monovalent straight-chain or branched-chain C1-C60 group optionally having one or more heteroatoms. Particularly, an alkyl is a C1-C45 group and more particularly, a C1-C30 group. As used herein, the term alkyl refers to C1-C4 group.

The terms “effective amount” and “effective use level” refer to a sufficient amount of functional active or solubilizing agent employed to provide desired performance attributes, stability, efficacy, product aesthetics, and the like.

The term, “functional active” as used herein refers to, e.g., chemically-active, pharmaceutically-active, or nutraceutically active ions, molecules, complexes, or polymers. It includes a personal care, cosmetic, pharmaceutical, and nutraceutical active compounds. In one embodiment, the functional active is a sunscreen containing UV-A and/or UV-B active organic or a mineral photoprotective agent that is water-soluble or liposoluble or insoluble in cosmetic solvents commonly employed.

The terms “personal care composition” and/or “cosmetics” herein refer to compositions intended for application on one or more parts of human body, including but not limited to skin, hair, lips, eyebrows, or eyelashes.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain the copolymer.

The term “photoprotective” as used herein refers to the ability of a functional active, preferably sunscreen active, to maintain its integrity when exposed to sunlight when combined with the cycloalkyl toluate based compound. A photo protective composition generally has an SPF (sunscreen protection factor) of at least 6. In another embodiment of the present application discloses at least 30, 45, 60 or 100 as suitable SPF for the sunscreen compositions. The SPF is duly defined in “A new substrate to measure sunscreen protection factors throughout the ultraviolet spectrum,” J. Soc. Cosmet. Chem., 40, 127-133, 1989 which is hereby incorporated by reference in its entirety.

The term “photoprotective composition” includes cosmetic compositions, personal care compositions, pharmaceutical compositions and/or nutraceutical compositions.

The term “photostable” as used herein refers to the ability of a molecule to remain intact with irradiation.

The minimal erythemal dose (MED) is defined as the lowest time interval or dosage of UV light irradiation sufficient to produce a minimal, perceptible erythema on unprotected skin (Wood et al., 2000; Wolf et al., 2001).

The term “skin” as used herein includes the skin on the face, neck, chest, back, abdomen, arms, hands, legs, feet, and scalp.

The term “sunscreen composition” refer to any composition intended for use on the human body for protection from harmful or undesirable radiations from sun rays.

What is described herein is a sunscreen composition comprising: avobenzene, a functional active, benzyl o-toluate, a stabilizing and solubilizing compound and at least one additive.

In a non-limiting embodiment, the present application discloses a sunscreen composition comprising: (i) about 0.01 wt. % to about 20 wt. % of avobenzone, (ii) about 0.01 wt. % to about 20 wt. % of benzyl o-toluate, and (iii) about 0.01 wt. % to about 95 wt. % of at least one additive.

In some embodiments, the sunscreen composition further comprises about 0.01 wt. % to about 25 wt. % at least one UV screening agents, physical sun blockers and combinations thereof.

Non-limiting examples of UV screening agents and/or physical sun blockers include but are not limited to organic and inorganic materials that scatter, absorb, and/or reflect radiation having a wavelength from about 100 nm to about 400 nm. In one particular and non-limiting embodiment of the present application discloses that the sun care composition protects UV-A, UV-B, and/or UV-C radiation. UV-A radiation includes longest wavelength range of from about 320 nm to about 400 nm within the UV spectrum, and consequently is the least energetic. UV-A radiation includes UV-A1 (from about 340 nm to about 400 nm) and UV-A2 (from about 320 nm to about 340 nm). UV-B radiation has shorter wavelengths, from about 290 nm to about 320 nm. UV-C radiation has the shortest wavelengths from about 200 nm to about 290 nm. Suitable UV actives (or UV filters) that can be selected for inclusion in the personal care compositions most likely will depend on local regulations. It is well known that commercially available sunscreens generally contain combinations of multiple sunscreen actives.

As used herein, UV screening agents and/or physical sun blockers are selected from the group including, but not limited to, octisalate, homosalate, octocrylene, benzopheneone-3, bis-ethylhexyloxyphenol methoxyphenyl triazine, butyloctyl salicylate, p-aminobenzoic acid and its derivatives, anthranilates, camphor analogues, cinnamic derivatives, dibenzoyl methanes, β-diphenylacrylate derivatives, salicylic derivatives, triazine derivatives, benzimidazole compounds, bis-benzoazolyl derivatives, methylene bis-(hydroxyphenylbenzotriazole) compounds, oxyethylene (25 mol) p-aminobenzoate, 2-ethylhexyl p-dimethylaminobenzoate, ethyl N-oxypropylene p-aminobenzoate, glycerol p-aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammunium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, β-(2-oxoborn-3-ylidene)-tolyl-4-sulfonic acid and soluble salts thereof, 3-(4′-sulfo)benzylidenbornan-2-one and soluble salts thereof, 3-(4′methylbenzylidene)-d,l-camphor, 3-benzylidene-d,l-camphor, benzene 1,4-di(3-methylidene-10-camphosulfonic) acid, urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl)anilino]-1,3,5-triazine, N-(2 and 4)-[(2-oxoborn-3-yliden)methyl]benzyl]-acrylamide, 1,4-bis-benzimidazolyl-phenylen-3,3′,5,5′-tetrasulfonic acid, the benzalmalonate-substituted polyorganosiloxanes, benzotriazole-substituted polyorganosiloxanes, dispersed 2,2′-methylene-bis-[6-(2H-benzotriazol-2-yl)-4-(1,1,3,3-tetramethylbutyl)phenol], solubilized 2,2′-methylene-bis-[6-(2H-benzotriazol-2-yl)-4-(methyl)phenol, cerium oxides, chromium oxides, cobalt oxides, iron oxides, red petrolatum, silicone-treated titanium dioxide, titanium dioxide, titanium dioxide (and) isopropyl titanium triisostearate, zirconium oxide, zinc oxide, zinc oxide (and) isopropyl titanium triisostearate, zinc oxide (and) triethoxycaprylylsilane, and derivatives and combinations thereof.

According to another embodiment, the present application discloses UV screening agents and/or physical sun blockers selected from the group consisting of octisalate, homosalate, octocrylene, benzopheneone-3, bis-ethylhexyloxyphenol methoxyphenyl triazine, butyloctyl salicylate, titanium dioxide, titanium dioxide (and) isopropyl titanium triisostearate, zirconium oxide zinc oxide, zinc oxide (and) isopropyl titanium triisostearate, zinc oxide (and) triethoxycaprylylsilane, and derivatives and combinations thereof.

According to another embodiment of the present application, the total amount of UV screening agents and/or physical sun blockers ranges from about 0.01 wt. % to about 5 wt. %, or from about 5 wt. % to about 10 wt. %, or 10 wt. % to about 15 wt. %, or from about 15 wt. % to about 20 wt. %, or from about 20 wt. % to about 25 wt. %, based on the total weight of the sunscreen composition.

In another specific embodiment, the composition of the present application comprises at least one additive selected from the group consisting of secondary polymers for improving water-resistance, oils, waxes, solvents, emulsifiers, preservatives, antioxidants, vitamins, perfumes, insect repellants, dyes, pigments, fillers, film formers, buffers, spreading agents, pearlizing agents, electrolytes, acids, bases, fatty substances, organic solvents, ionic or nonionic, hydrophilic or lipophilic thickeners, softeners, humectants, opacifiers, emollients, fragrances, anionic surfactants, cationic surfactants, nonionic surfactants, zwitterionic surfactants, amphoteric surfactants, active agents, polymers, propellants, antioxidants, agents for combating free radicals, α-hydroxy acids, moisturizing agents, anti-inflammatory, colorants, pharmaceutically or dermatologically or cosmetically acceptable excipients, skin care or hair care agents, hair styling agents, hair fixative agents, structurants, gelling agents, viscosity modifiers, electrolytes, pH adjusting agents, organo silicones compounds, anti-dandruff agents, antifoaming agents, anti-frizz agents, penetrants, conditioning agents, chelating agents, antimicrobial agents, UV absorbers, natural extracts, carriers, diluents, solvents, pharmaceutical actives, lubricants, combing aids, plasticizers, solubilizers, neutralizing agents, vapor pressure suppressants, bleaching agents, hydrating agents, cosmetic adjuvants and/or additives, protectants, and mixtures thereof.

According to some embodiment of the present invention, the additive is selected from the group consisting of emulsifiers, humectants, emollients, preservatives, buffering agents and/or pH adjusters, rheology modifiers, liquid carriers, film forming and/or water proofing agent, chelating agents, siloxanes, emulsion stabilizers, fatty acid esters, solvents, propellants, surfactants, skin-feel additives, anti-aging agents, exfoliating agents, defoamers, dyes, fragrances, waxes, conditioners, lubricants, antibiotics, viscosity synergists, clarity synergists, antioxidants, and combinations thereof.

As used herein, emulsion stabilizers are selected from the group comprising salts of alkaline earth metals including but not limited to sodium chloride, potassium chloride or combinations thereof.

Non-limiting examples of emulsifiers include ethoxylated fatty acids, ethoxylated glyceryl esters, ethoxylated oils, ethoxylated sorbitan esters, fatty esters, PEG esters and polyglycerol esters.

As used herein, emulsifiers are selected from the group consisting of C16-C18 alkyl alcohol ethoxylate, glyceryl stearate, glyceryl stearate SE, glyceryl Stearate (and) PEG-100 stearate, dimethicone (and) dimethicone/PEG-10/15 crosspolymer, cetyl PEG/PPG-10/1 Dimethicone, polyglyceryl-4 isostearate (and) cetyl PEG/PPG-10/1 dimethicone (and) hexyl laurate, lauryl PEG-9 polydimethylsiloxyethyl dimethicone, glyceryl laurate, glyceryl dilaurate, glycol distearate, sorbitan trioleate, glycol stearate, sorbitan sesquioleate, lecithin, sorbitan oleate, sorbitan stearate, sorbitan isostearate, oleth-2, steareth-2, calcium stearoyl lactylate, ceteth-2, PEG-4 dilaurate, sorbitan stearate (and) sucrose cocoate, methyl glucose sesquistearate, sucrose stearate, PEG-10 sunflower glycerides, sodium stearoyl lactylate, sorbitan laurate, PEG-40 sorbitan peroleate, lecithin, laureth-4, PEG-7 glyceryl cocoate, PEG-20 almond glycerides, linoleamide DEA, PEG-25 hydrogenated castor oil, cetearyl glucoside, polysorbate 85, glyceryl stearate (and) PEG-100 stearate, stearamide MEA, oleth-10/polyoxyl 10 oleyl ether NF, oleth-10, oleth-20, ceteth-10, PEG-8 laurate, cocamide MEA, polysorbate 60, PEG-60 almond glycerides, isosteareth-20, lauramide DEA, PEG-30 dipolyhydroxystearate, polysorbate 80, PEG-20 methyl glucose sesquistearate, oleth-20, steareth-21, isoceteth-20, polysorbate 20, laureth-23, PEG-100 stearate, PEG-80 sorbitan laurate, potassium cetyl phosphate, cetearyl alcohol, diacetyl phosphate, ceteth-10 phosphate, and combinations thereof. In some embodiments, the present application discloses suitable range of emulsifiers that can be varied from about 0.01 wt. % to about 5 wt. %, or from about 5 wt. % to about 10 wt. %, or 10 wt. % to about 15 wt. %, based on the total weight of the sunscreen composition.

As used herein, humectants are selected from the group consisting of glycerin, polyethylene glycol, propylene glycol, ethylene glycol, diethylene glycol, propylene glycol, methyl alcohol, ethyl alcohol, propyl alcohol, isopropyl alcohol, glycerol, sorbitol, and combinations thereof. In some embodiments, the present application discloses suitable range of humectants that can be varied from about 0.01 wt. % to about 5 wt. %, or from about 5 wt. % to about 10 wt. %, based on the total weight of the sunscreen composition.

Non-limiting examples of emollient include lauryl lactate, C12-15 Alkyl lactate, methyl glucose sesquistearate and PEG-20 methyl glucose sesquistearate. As used herein, emollients are selected from the group consisting of cetyl alcohol, isocetyl alcohol, isodecyl neopentanoate, isostearyl neopentanoate and isocetyl stearyl stearate, diisopropyl adipate, lauryl lactate, C12-15 alkyl lactate, dicaprylyl carbonate, isodecyl salicylate, glycerin, glycerin (and) glyceryl acrylate/acrylic acid copolymer (and) PVM/MA copolymer, octyl palmitate, diisopropyl adipate, and combinations thereof. In some embodiments, the present application discloses suitable range of emollients that can be varied from about 0.01 wt. % to about 5 wt. %, or from about 5 wt. % to about 10 wt. %, based on the total weight of the sunscreen composition.

Non-limiting examples of suitable preservatives used in the present application include benzoic acid, sorbic acid, dehydroacetic acid, diazolidinyl ureas, imidazolidinyl ureas, salicylic acid, piroctone olamine, DMDM hydantoin, IPBC (iodopropynyl butylcarbamate), triclosan, bronopol, formaldehyde, isothiazolinones, nitrates/nitrites, parabens, phenoxyethanol, potassium sorbate, sodium benzoate, sulphites, sulphur dioxide, and blends thereof.

As used herein, preservatives are selected from the group consisting of propylene glycol (and) diazolidinyl urea (and) methylparaben (and) propylparaben, methyl 4-hydroxybenzoate, phenoxyethanol (and) benzoic acid (and) dehydroacetic acid, 1,2-Hexanediol, lower alkyl esters of para-hydroxybenzoates (parabens) including methylparaben, propylparaben, isobutylparaben, phenoxyethanol (and) methylparaben (and) ethylparaben (and) propylparaben; benzyl alcohol, phenoxyethanol, benzoic acid, caprylyl glycol (and) phenoxyethanol and combinations thereof. In some embodiments, the present application discloses suitable range of preservatives that can be varied from about 0.01 wt. % to about 2.50 wt. %, or from about 2.50 wt. % to about 5 wt. %, based on the total weight of the sunscreen composition.

Non-limiting examples of buffering agents used in the present application include alkali or alkali earth carbonates, phosphates, bicarbonates, citrates, borates, acetates, acid anhydrides, succinates, and the like, such as sodium phosphate, citrate, borate, acetate, bicarbonate, and carbonate.

Buffering agents and/or pH adjusters are selected from the group consisting of triethanolamine, sodium diacetate, sodium acetate, sodium succinate, disodium succinate, sodium citrate, sodium formate, sodium hydroxide, sodium or ammonium salts, and combinations thereof.

Rheology modifiers are selected from the group consisting of polyacrylates, acrylate copolymers, acrylic acid/vinylpyrrolidone cross polymer, acrylates (and) C10-C30 alkyl acrylate cross polymer, methyl cellulose, ethyl cellulose, hydroxyethyl cellulose, hydroxypropylmethyl cellulose, stearalkonium hectorite (and) caprylic/capric triglyceride (and) propylene carbonate, and combinations thereof. In some embodiments, the present application discloses suitable range of rheology modifiers that can be varied from about 0.01 wt. % to about 2.50 wt. %, or from about 2.50 wt. % to about 5 wt. %, based on the total weight of the sunscreen composition.

The liquid carriers are selected from the group consisting of alcohols, water, natural oils including but not limited to coconut oil, olive oil, sunflower seed oil, jojoba oil, shea butter, almond oil, grapeseed oil, and any combination thereof. In some embodiments, the present application discloses suitable range of liquid carriers that can be varied from about 0.01 wt. % to about 10 wt. %, or from about 10 wt. % to about 20 wt. %, or from about 20 wt. % to about 30 wt. %, or from about 30 wt. % to about 40 wt. %, or from about 50 wt. % to about 60 wt. %, or from about 60 wt. % to about 70 wt. %, based on the total weight of the sunscreen composition.

Chelating agents are selected from the group consisting of Tetrasodium EDTA, disodium pyrophosphate, dipotassium pyrophosphate, tetrapotassium pyrophosphates, tetrasodium pyrophosphates, disodium ethane-1-hydroxy-1,1-diphosphonate (EHDP), methanediphosphonic acid, 2-phosphonobutane-1,2,4-tricarboxylic acid, citric acid, sodium citrate, potassium citrate, tartaric acid, disodium calcium ethylene diamine tetraacetic acid, disodium ethylene diamine tetraacetate, disodium ethylene diamine tetraacetic acid, gluconic acid, methyl vinyl ether and maleic anhydride (PVM/MA) copolymers, polyacrylic acid, polyitaconic acid and combinations thereof. In some embodiments, the present application discloses suitable range of chelating agents that can be varied from about 0.01 wt. % to about 2.50 wt. %, or from about 2.50 wt. % to about 5 wt. %, based on the total weight of the sunscreen composition.

The film forming and/or water proofing agents are selected from the group consisting of vinyl acetate (and) butyl maleate (and) isobornyl acrylate copolymer, vinylpyrrolidone (and) eicosene copolymer, vinylpyrrolidone (and) acrylates (and) lauryl methacrylate copolymer; vinyl pyrrolidone (and) 1-triacontane copolymer, vinylpyrrolidone (and) hexadecene copolymer, poly (butylated vinylpyrrolidone), vinylpyrrolidone (and) hexadecene copolymer, acrylates (and) C12-C22 alkylmethacrylate copolymer, Tricontanyl polyvinylpyrrolidone, hydroxyethyl cellulose, and combinations thereof. In some embodiments, the present application discloses suitable range of film forming and/or water proofing agents that can be varied from about 0.01 wt. % to about 2.50 wt. %, or from about 2.50 wt. % to about 5 wt. %, based on the total weight of the sunscreen composition.

Suitable siloxanes for the purposes of this application are selected from the group consisting of dimethylpolysiloxane, cyclopentasiloxane, cyclopentasiloxane (and) dimethylpolysiloxane (and) vinyltrimethylsiloxysilicate cross polymer, polyalkyl siloxanes, phenyl trimethicone, polyaryl siloxanes, poly alkyl aryl siloxane, an organic functional group modified poly-organic siloxane and combinations thereof.

Accordingly, the additive used in the present application is present in an amount of from about 0.1% wt./wt. to about 95% wt./wt. of the total composition. Other non-limiting ranges for additive includes 0.1% to 5%, 5% to 10%, 10% to 15%, 15% to 20%, 20% to 25% or 25% to 30%, 30% to 40%, 40% to 50%, 50% to 60%, 60% to 70%, 70% to 80%, 80% to 90% and 90% to 95% wt./wt. of the total composition.

In some embodiments, the suitable range of avobenzone for the present application can be varied from about 0.01 wt. % to about 5 wt. %, or from about 5 wt. % to about 10 wt. %, or 10 wt. % to about 15 wt. %, or from about 15 wt. % to about 20 wt. %, based on the total weight of the sunscreen composition.

In some embodiments, the suitable range of benzyl o-toluate for the present application can be varied from about 0.01 wt. % to about 5 wt. %, or from about 5 wt. % to about 10 wt. %, or 10 wt. % to about 15 wt. %, or from about 15 wt. % to about 20 wt. %, based on the total weight of the sunscreen composition.

In another non-limiting embodiment, the suitable surfactant for the purpose of the present application is selected from the group consisting of anionic, zwitterionic, amphoteric, nonionic, or cationic surfactant, or combinations thereof.

Nonionic surfactants can be broadly defined as compounds containing a hydrophobic moiety and a nonionic hydrophilic moiety. Examples of the hydrophobic moiety can be alkyl, alkyl aromatic, dialkyl siloxane, polyoxyalkylene, and fluoro-substituted alkyls. Examples of hydrophilic moieties are polyoxyalkylenes, phosphine oxides, sulfoxides, amine oxides, and amides. Nonionic surfactants such as those marketed under the trade name Surfynol® are also useful in this invention. Nonionic surfactants useful herein include compounds produced by the condensation of alkylene oxide groups (hydrophilic in nature) with an organic hydrophobic compound which maybe aliphatic or alkylaromatic in nature. Non-limiting examples of suitable nonionic surfactants include: poloxamers (sold under the trade name Pluronic® by BASF Corporation), polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and blends thereof.

Cationic surfactants useful in vehicle systems of the compositions of the present invention contain amino or quaternary ammonium hydrophilic moieties which are positively charged when dissolved in the aqueous composition of the present invention. Non-limiting examples of suitable cationic surfactants include derivatives of aliphatic quaternary ammonium compounds having at least one long alkyl chain containing from about 8 to about 18 carbon atoms, such as, lauryl trimethylammonium chloride, cetyl pyridinium chloride, cetyl trimethylammonium bromide, di-isobutylphenoxyethyl-dimethylbenzylammonium chloride, coconut alkyl-trimethylammonium nitrite, cetyl pyridinium fluoride, and blends thereof. Further suitable are quaternary ammonium fluorides having detergent properties such as compounds described in U.S. Pat. No. 3,535,421. Certain cationic surfactants may act as germicides in the compositions disclosed herein.

Cationic surfactants can have a hydrophobe that carries a positive charge or that is uncharged at pH values close to neutrality or lower, such as alkylamines, alkyl imidazolines, ethoxylated amines, and quaternary ammonium compounds. Alkylamines can be salts of primary, secondary and tertiary fatty C₁₂-C₂₂ alkylamines, substituted or unsubstituted, and substances sometimes referred to as “amidoamines”. Non-limiting examples of alkylamines and salts thereof include dimethyl cocamine, dimethyl palmitamine, dioctylamine, dimethyl stearamine, dimethyl soyamine, soyamine, myristyl amine, tridecyl amine, ethyl stearylamine, N-tallowpropane diamine, ethoxylated stearylamine, dihydroxy ethyl stearylamine, arachidylbehenylamine, dimethyl lauramine, stearylamine hydrochloride, soyamine chloride, stearylamine formate, N-tallowpropane diamine dichloride, and amodimethicone (INCI name for a silicone polymer and blocked with amino functional groups, such as aminoethylamino propylsiloxane). Non-limiting examples of amidoamines and salts thereof include stearamido propyl dimethyl amine, stearamidopropyl dimethylamine citrate, palmitamidopropyl diethylamine, and cocamidopropyl dimethylamine lactate. Other cationic surfactants include distearyldimonium chloride, dicetyldimonium chloride, guar hydroxypropyltrimonium chloride, and the like. At low pH, amine oxides may protonate and behave similarly to N-alkyl amines. Non-limiting examples of alkyl imidazolines include alkyl hydroxyethyl imidazoline, such as stearyl hydroxyethyl imidazoline, coco hydroxyethyl imidazoline, ethyl hydroxymethyl oleyl oxazoline, and the like. Non-limiting examples of ethyoxylated amines include PEG-cocopolyamine, PEG-15 tallow amine, quaternium-52, and the like.

Quaternary ammonium compounds can be selected from monomeric or polymeric materials containing at least one nitrogen atom that is linked covalently to four alkyl and/or aryl substituents, and the nitrogen atom remains positively charged regardless of the environmental pH. Quaternary ammonium compounds comprise a large number of substances that are used extensively as surfactants, conditioners, antistatic agents, and antimicrobial agents and include, alkylbenzyldimethyl ammonium salts, alkyl betaines, heterocyclic ammonium salts, and tetraalkylammonium salts. Long-chain (fatty) alkylbenzyldimethyl ammonium salts are preferred as conditioners, as antistatic agents, and as fabric softeners, discussed in more detail below. Other quaternary ammonium compounds include quaternary ammonium silicones. While various quaternary ammonium compounds are listed for a specific purpose, one of ordinary skill will recognize that the quaternary ammonium compounds described here and throughout the specification can serve more than one function.

Non-limiting examples of quaternary ammonium compounds include, PQ-2, PQ-4, PQ-5, PQ-6, PQ-7, PQ-8, PQ-9, PQ-10, PQ-11, PQ-14, PQ-16, PQ-17, PQ-18, PQ-19, PQ-20, PQ-21, PQ-22, PQ-24, PQ-27, PQ-28, PQ-29, PQ-31, PQ-32, PQ-37, PQ-39, PQ 41, PQ-42, PQ-44, PQ-46, PQ-47, PQ-48, PQ-49, PQ-50, PQ-55, PQ-69 and other quaternary ammonium compounds are listed in the CTFA Cosmetic Ingredient Handbook, First Edition, on pages 41-42, incorporated herein by reference, and are described in the “History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

Non-limiting examples of alkylbenzyldimethylammonium salts include stearalkonium chloride, benzalkonium chloride, quaternium-63, olealkonium chloride, didecyldimonium chloride, and the like. Alkyl betaine compounds include alkylamidopropyl betaine, alkylamidopropyl hydroxysultaine, and sodium alkylamido propyl hydroxyphostaine. Non-limiting examples of alkyl betaine compounds include oleyl betaine, coco-betaine, cocoamidopropyl betaine, coco-hydroxy sultaine, coco/oleamidopropyl betaine, coco-sultaine, cocoamidopropylhydroxy sultaine, and sodium lauramidopropyl hydroxyphostaine. Heterocyclic ammonium salts include alkylethyl morpholinium ethosulfate, isostearyl ethylimidonium ethosulfate, and alkylpyridinium chlorides, and are generally used as emulsifying agents. Non-limiting examples of heterocyclic ammonium salts include cetylpyridinium chloride, isostearylethylimidonium ethosulfate, and the like. Non-limiting examples of tetraalkylammonium salts include cocamidopropyl ethyldimonium ethosulfate, hydroxyethyl cetyldimonium chloride, quaternium-18, and cocodimonium hyroxypropyl hydrolyzed protein, such as hair keratin, and the like.

Zwitterionic surfactants are exemplified by those which can be broadly described as derivative of aliphatic quaternary ammonium, phosphonium, and sulfonium compounds, which can be broadly described as derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains as anionic water-solubilizing group, e.g., carboxy, sulfonate, sulfate, phosphate, or phosphonate. Non-limiting examples of suitable zwitterionic surfactants include betaines and derivatives of aliphatic quaternary ammonium compounds in which the aliphatic radicals can be straight chain or branched, and which contain an anionic water-solubilizing group, e.g., carboxy, sulfonate, sulfate, phosphate, or phosphonate.

Examples of amphoteric surfactants which can be used in the vehicle systems of the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglycerlde sulfonates are non-limiting examples of anionic surfactants of this type. Non-limiting examples of suitable anionic surfactants include: sarcosinates, taurates, isethionates, sodium lauryl sulfoacetate, sodium laureth carboxylate, and sodium dodecyl benzenesulfonate. Also suitable are alkali metal or ammonium salts of surfactants such as the sodium and potassium salts of the following: lauroyl sarcosinate, myristoyl sarcosinate, palmitoyl sarcosinate, stearoyl sarcosinate, and oleoyl sarcosinate. Other surfactants such as fluorinated surfactants may also be incorporated within the compositions of the invention.

Anionic surfactants include substances having a negatively charged hydrophobe or that carry a negative charge when the pH is elevated to neutrality or above, such as acylamino acids, and salts thereof, for example, acylglutamates, acyl peptides, sarcosinates, and taurates; carboxylic acids, and salts thereof, for example, alkanolic acids and alkanoates, ester carboxylic acids, and ether carboxylic acids; phosphoric acid ester and salts thereof; sulfonic acids and salts thereof, for example, acyl isethionates, alkylaryl sulfonates, alkyl sulfonates, and sulfosuccinates; and sulfuric acid esters, such as alkyl ether sulfates and alkyl sulfates.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglycerlde sulfonates are non-limiting examples of anionic surfactants of this type. Non-limiting examples of suitable anionic surfactants include: sarcosinates, taurates, isethionates, sodium lauryl sulfoacetate, sodium laureth carboxylate, and sodium dodecyl benzenesulfonate. Also suitable are alkali metal or ammonium salts of surfactants such as the sodium and potassium salts of the following: lauroyl sarcosinate, myristoyl sarcosinate, palmitoyl sarcosinate, stearoyl sarcosinate, and oleoyl sarcosinate. Other surfactants such as fluorinated surfactants may also be incorporated within the compositions of the invention.

Non-limiting examples of anionic surfactants include mono-basic salts of acylglutamates that are slightly acidic in aqueous solution, such as sodium acylglutamate and sodium hydrogenated tallow glutamate; salts of acyl-hydrolyzed protein, such as potassium, palmitoyl hydrolyzed milk protein, sodium cocoyl hydrolyzed soy protein, and TEA-abietoyl hydrolyzed collagen; salts of acyl sarcosinates, such as ammonium myristoyl sarcosine, sodium cocoyl sarcosinate, and TEA-lauroyl sarcosinate; salts of sodium methyl acyltaurates, such as sodium lauroyl taurate and sodium methyl cocoyl taurate; alkanoic acids and alkanoates, such as fatty acids derived from animal and vegetable glycerides that form water-soluble soaps and water-insoluble emulsifying soaps, including sodium stearate, aluminum stearate, and zinc undecylenate; ester carboxylic acids, such as dinonoxynol-9-citrate; salts of acyl lactylates such as calcium stearoyl lactylate and laureth-6 citrate; ethercarboxylic acids derived from ethyoxylated alcohols or phenols having varying lengths of polyoxyethylene chains, such as nonoxynol-8 carboxylic acid, and sodium trideceth-13 carboxylate; mono and di-esters of phosphoric acid and their salts, such as phospholipids, dilaureth-4-phosphate, DEA-oleth-10 phosphate and triethanolamine lauryl phosphate; salts of acylisethionate, such as sodium cocoyl isethionate; alkylarylbenzene sulfonates, such as α-olefin sulfonate (AOS) and alkali metal, alkaline earth metal, and alkanolamine salts thereof, and sodium dodecylbenzene sulfonate; alkyl sulfonates, such as sodium C₁₂-C₁₄ olefin sulfonate, sodium cocomonoglyceride sulfonate, sodium C₁₂-C₁₅ pareth-15 sulfonate, and sodium lauryl sulfoacetate; sulfosuccinates, such as mono and di-esters of sulfosuccinic acid, salts thereof and alkoxylated alkyl and alkylamido derivatives thereof, such as di-C₄-C₁₀ alkyl sodium sulfosuccinate, disodium laureth sulfosuccinate, disodium oleamido MEA-sulfosuccinate, and disodium C₁₂-C₁₅ pareth sulfosuccinate; alkyl ether sulfates, such as sodium and ammonium lauryl ether sulfate (having about 1 to about 12 moles ethylene oxide); alkyl sulfates, such as sodium, ammonium and triethanolamine salts of C₁₂-C₁₈ alkylsulfates, sodium C₁₂-C₁₄ olefin sulfates, sodium laureth-6 carboxylate, sodium C₁₂-C₁₈ pareth sulfate, and the like.

Also suitable surfactants are selected from the following commercial products: (1) Alkanolamides, under the trade names Amidex and Schercomid; amido-amines, under the trade names Katemul and Schercodine; amine oxides, under the trade names Chemoxide™ and Schercamox™; amphoterics, under the trade names Chembetaine™, Schercotaine™ and Schercoteric™; imidazolines, under the trade name Schercozoline™; pearlizing agents, under the trade name Quickpearl™; performance concentrates, under the trade names Sulfochem™ and Chemoryl™; soaps (potassium cocoate and potassium soyate); specialty ethoxylates, under the trade name Chemonic™; specialty quats under the trade names Quatrex and Schercoquat, sulfates, under the trade name Sulfochem; and sulfosuccinates, under the trade name Chemccinate™ from Lubrizol. (2) Avaniel, Cremaphore®, Jordapan®, and Pluracar® from BASF Corp. [00190] (3) Miracare® SLB, Mackam® Bab, Mackanate® Ultra SI, Miranof Ultra, and Miracare® Plaisant from Rhodia.] (4) Stepan® Pearl 2, Stepan® Pearl 4, Stepan® Pearl Series, Neobee® M-20, Stepan® PTC, Amphosol® 2CSF, Steol®, Stepan-Mild® GCC, Stepan® SLI\hat{ }FB, Stepanol® AM, Stepanol® PB, Alpha-Step® BSS-45, Bio-Terge® 804, Stepan-Mild® L3, Stepan® SLL-FB, Stepan® SSL-CG, and Stepanol® CFAS-70 from Stepan Company.

Also, suitable surfactants are selected from those described in the book Surfactants in Personal Care Products and Decorative Cosmetics, Third Edition, 2006, CRC Press, the disclosure is incorporated hereby in its entirety by reference.

In some embodiments, the present application discloses suitable ranges of surfactant that can be varied from about 0.1 wt. % to about 1 wt. %, or from about 1 wt. % to about 2.5 wt. %, or from about 2.5 wt. % to about 5 wt. % or from about 5 wt. % to about 10 wt. %, or 10 wt. % to about 15 wt. %, or from about 15 wt. % to about 20 wt. %, or from about 20 wt. % to about 25 wt. %, or from about 25 wt. % to about 30 wt. % based on the total weight of the aqueous personal care or home care compositions.

In another specific embodiment, the present application provides a sunscreen composition formulated into an oil-in-water emulsion, a water-in-oil emulsion, an oil-water-oil emulsion, a water-oil-water emulsion, a water-in-silicone emulsion, an oily solution, a lipid fusion, a hydro-alcoholic gel, an anhydrous formulation, an anhydrous spray, an anhydrous gel, an anhydrous dry gel, an aqueous gel, a gel cream, a SPF 20 cream, a SPF 30 cream, an alcoholic solution, a hydro-alcoholic solution, a milk, a lotion, a powder, a stick, a roll-on, a mist, a wipe, wax, a mousse, an aerosol, a balm, a patch, a pomade, a pump spray, a solution, a towelette, paste, powder or a spray.

In some embodiments, the sunscreen composition is in the form of a lotion, an oil-in-water emulsion, a water-in-oil emulsion, a spray, a water-in-silicone emulsion, an oily solution, a lipid fusion, a hydro-alcoholic gel, an anhydrous gel, an aqueous gel, an alcoholic solutions, or a hydro-alcoholic solution.

In some embodiments, non-limiting examples of the sunscreen composition include a sun care composition, face care composition, lip care composition, eye care composition, skin care composition, after-sun care composition, body care composition, nail care composition, anti-aging composition, deodorant composition, hair care composition, conditioning composition, color cosmetic composition, color-protection composition, self-tanning composition, and foot care composition.

In another non-limiting embodiment, the present application provides a sunscreen composition formulated, for example into products for application to the lips, hair, face, cheeks, neck, area around the eyes, full hands, and body area.

Non-limiting examples of sunscreen composition of the present application include but not limited to, lotions, creams, ointments, gels, W/O emulsions, O/W emulsions, milks, suspensions, micro emulsions, dispersions, microencapsulated products, balms, pastes, peels, aerosols, mousses, waxes, pomades, or putties

According to some embodiments, the present application discloses a solubilizer comprising benzyl o-toluate that effectively solubilizes and photostabilizes avobenzone in a sunscreen composition with a good odor.

According to some embodiments, the present application discloses a solubilizer comprising benzyl o-toluate, that effectively solubilizes bis-ethylhexyloxyphenol methoxyphenyl triazine in a sunscreen composition with a good odor.

A non-limiting embodiment of the present application discloses a method for solubilizing and photostabilizing avobenzone in a sunscreen composition comprising avobenzone, the method comprising adding about 0.01 wt. % to about 20 wt. % of benzyl o-toluate to the composition to stabilize the avobenzone against photodegradation and to effectively solubilize avobenzone.

Another non-limiting embodiment of the present application discloses a method for protecting the skin against UV radiation comprising, applying to the skin an effective amount of a sunscreen composition comprising: (i) about 0.01 wt. % to about 20 wt. % of avobenzone, (ii) about 0.01 wt. % to about 20 wt. % of benzyl o-toluate, and (iii) about 0.01 wt. % to about 95 wt. % of at least one additive.

In another non-limiting embodiment of the present application, the skin includes human skin or animal skin.

Further, certain aspects of the present application are illustrated in detail by way of the following examples. The examples are given herein for illustration of the application and are not intended to be limiting thereof.

### EXAMPLES

### Example-1: SPF 30 Cream

Method of preparation of SPF 30 cream as per formulation of Table-1: Ingredients of Phase A were combined, mixed well, and heated to a temperature of 80° C. Ingredients of Phase B were also mixed separately by stirring and heating to a temperature of 80° C. Phase B mixture was then added to Phase A and homogenized well. Phase C was then added to the mixture while cooling. Phase D was then added to the batch at a temperature of 40° C. and cooled to room temperature with stirring.

TABLE 1
Formulation of SPF 30 cream.

| | |
|---|---|
| Ingredients | % w/w |

Phase A

| | |
|---|---|
| Water | 62.15 |
| Carbomer | 0.40 |

Phase B

| | |
|---|---|
| Avobenzone | 1.00 |
| Octisalate | 5.00 |
| Homosalate | 10.0 |
| Octocrylene | 0.80 |
| Benzopheneone-3 | 4.00 |
| Bis-ethylhexyloxyphenol methoxyphenyl triazine | 1.00 |
| Glyceryl Stearate (and) laureth-23 | 3.00 |
| Ceteareth-20 | 1.50 |
| Benzyl o-toluate | 5.00 |

Phase C

| | |
|---|---|
| Water | 5.00 |
| Triethanolamine | 0.40 |

Phase D

| | |
|---|---|
| Propylene Glycol (and) Diazolidinyl Urea (and) methylparaben | 0.75 |
| (and) propylparaben | |
| Total | 100 |

### Example-2: Harmonizing Lotion

Method of preparation of Harmonizing lotion as per formulation of Table-2: Ingredients of Phase A were combined, mixed well, and heated to a temperature of 80° C. Ingredients of Phase B were also mixed separately by stirring and heating to a temperature of 80° C. Phase B mixture was then added to Phase A and homogenized well. Phase C was then added to the mixture while cooling. Phase D was then added to the batch at a temperature of 40° C. and cooled to room temperature with stirring.

TABLE 2
Formulation of Harmonizing lotion.

| | |
|---|---|
| Ingredients | % w/w |

Phase A

| | |
|---|---|
| Water | 62.50 |
| Glycerin | 3.00 |
| Disodium EDTA | 0.10 |
| Triethanolamine | 0.50 |
| Acrylic Acid/Vinylpyrrolidone Cross polymer | 0.70 |

Phase B

| | |
|---|---|
| Dimethicone | 0.50 |
| PEG-20 Methyl Glucose Sesquistearate | 2.50 |
| Methyl Glucose Sesquistearate | 0.50 |
| Cetyl Alcohol | 1.50 |
| Isodecyl neopentanoate | 3.00 |
| Avobenzone | 1.00 |
| Octisalate | 5.00 |
| Octocrylene | 2.00 |
| Bis-ethylhexyloxyphenol methoxyphenyl triazine | 3.00 |
| Benzyl o-toluate | 5.00 |

Phase C

| | |
|---|---|
| Cyclopentasiloxane | 5.00 |
| Cyclopentasiloxane (and) Dimethicone/ | 1.00 |
| Vinyltrimethylsiloxysilicate Cross polymer | |

Phase D

| | |
|---|---|
| Phenoxyethanol (and) benzoic Acid (and) | 0.75 |
| Dehydroacetic Acid | |
| Total | 100 |

### Example-3: HydroSheer™ Spray

Method of preparation of HydroSheer™ spray as per formulation of Table-3: Ingredients of Phase A were mixed well until completely dissolved. Ingredients of Phase B were combined and mixed well in a separate vessel with mild heating until crystals were completely dissolved. Phase B mixture was then added to Phase A and continued stirring until homogenized well.

TABLE 3
Formulation of HydroSheer ™ spray.

| | |
|---|---|
| Ingredients | % w/w |

Phase A

| | |
|---|---|
| Ethanol | 44.00 |
| VA/Butyl Maleate/Isobornyl Acrylate Copolymer | 2.00 |

Phase B

| | |
|---|---|
| Avobenzone | 3.00 |
| Octisalate | 5.00 |
| Homosalate | 10.00 |
| Octocrylene | 8.00 |
| Bis-ethylhexyloxyphenol methoxyphenyl triazine | 2.00 |
| Benzyl o-toluate | 3.00 |
| Diisopropyl Adipate | 5.00 |
| Lauryl Lactate | 5.00 |
| C12-15 Alkyl Lactate | 5.00 |
| Dicaprylyl Carbonate | 4.00 |
| Isodecyl Salicylate | 3.00 |
| Total | 100 |

### Example 4: Emulsion

Method of preparation of Emulsion as per formulation of Table-4: Ingredients of Phase A were combined, mixed well, and heated to a temperature of 80° C. Ingredients of Phase B were also mixed separately by stirring and heating to a temperature of 80° C. Phase B mixture was then added to Phase A and homogenized well. Phase C was then added to the mixture while cooling.

TABLE 4
Formulation of Emulsion.

| | |
|---|---|
| Ingredients | % w/w |

Phase A

| | |
|---|---|
| Purified water | 54.60 |
| Glycerin and Glyceryl Acrylate/Acrylic Acid | 5.00 |
| Copolymer and PVM/MA Copolymer | |
| Tetrasodium EDTA | 0.10 |
| Acrylic Acid/Vinylpyrrolidone cross polymer | 0.30 |
| Phenoxyethanol (and) Benzoic Acid (and) | 1.00 |
| Dehydroacetic Acid | |
| methyl 4-hydroxybenzoate | 0.30 |

Phase B

| | |
|---|---|
| Potassium Cetyl Phosphate | 1.50 |
| Ethylhexyl Triazone | 5.00 |
| Avobenzone | 3.00 |
| Bis-ethylhexyloxyphenol methoxyphenyl triazine | 3.00 |
| Octisalate | 5.00 |
| Homosalate | 10.00 |
| Benzyl o-toluate | 5.00 |
| Dimethicone | 2.00 |
| Behenyl Alcohol and polyglycerol-10 Pentastearate | 1.50 |
| and Sodium Stearoyl Lactate | |
| Antaron/Ganex Sensory | 0.70 |

Phase C

| | |
|---|---|
| NaOH 10% Solution | 2.00 |
| Total | 100 |

### Example-5: Test Emulsion

Method of preparation of Emulsion as per formulation of Table-5: Ingredients of Phase A were combined, mixed well, and heated to a temperature of 80° C. Ingredients of Phase B were also mixed separately by stirring and heating to a temperature of 80° C. Phase B mixture was then added to Phase A and homogenized well. Phase C was then added to the mixture while cooling.

TABLE 5
Formulation of Test emulsion.

| | |
|---|---|
| Ingredient | % w/w |

Phase A

| | |
|---|---|
| DI Water | 58.00 |
| Propylene Glycol | 2.00 |
| Carbomer 980 | 0.30 |
| Phenoxyethanol (and) Methylparaben (and) | 1.00 |
| Ethylparaben (and) Propylparaben | |

Phase B

| | |
|---|---|
| Avobenzone | 3.00 |
| Octisalate | 5.00 |
| Bis-ethylhexyloxyphenol methoxyphenyl | 3.00 |
| triazine | |
| Ethylhexyl Triazone | 5.00 |
| Homosalate | 10.00 |
| Glyceryl Stearate (and) PEG-100 Stearate | 4.00 |
| Benzyl o-toluate | 7.00 |
| Vinylpyrrolidone/Eicosene Copolymer | 1.00 |

Phase C

| | |
|---|---|
| NAOH 10% Solution | 0.70 |
| Total | 100 |

### Example-6: Water-in-Silicone Mineral (Zinc and Titanium) Emulsion

Method of preparation of Water-in-Silicone mineral (zinc and titanium) emulsion as per formulation of Table-6: Ingredients of Phase A were pre-weighted, mixed, and heated to a temperature of 40-45° C. The mixture was then homogenized until uniform and subsequently cooled down to a temperature of 30-35° C. Ingredients of Phase C were added to a separate beaker and mixed until clear while heating to a temperature of 65° C. It was then cooled down to a temperature of 30-35° C. Ingredients of Phase B were pulverized and added to Phase C while mixing at moderate high speed. Phase A mixture was then added to mixture of Phase B and Phase C slowly while mixing at moderate to high speed for about 10 minutes. It was then switched to slow sweep and cooled down to room temperature.

TABLE 6
Formulation of Water-in-Silicone mineral
(zinc and titanium) emulsion.

| | |
|---|---|
| Ingredients | % w/w |

Phase A

| | |
|---|---|
| DI water | 47.50 |
| Glycerin | 5.00 |
| Hydroxyethylcellulose | 0.50 |
| Sodium Chloride | 0.50 |
| Caprylyl Glycol, Phenoxyethanol | 1.50 |

Phase B

| | |
|---|---|
| Zinc Oxide (and) Isopropyl Titanium Triisostearate | 8.00 |
| Titanium dioxide (and) Isopropyl Titanium Triisostearate | 8.00 |

Phase C

| | |
|---|---|
| Dimethicone(and)Dimethicone/PEG-10/15 Crosspolymer | 2.50 |
| Lauryl PEG-9 Polydimethylsiloxyethyl Dimethicone | 3.00 |
| Benzyl o-toluate | 5.00 |
| Phenyl Trimethicone | 4.00 |
| Octyl Palmitate | 5.00 |
| Stearalkonium Hectorite & Caprylic/capric Triglyceride & | 2.50 |
| Propylene Carbonate | |
| Tricontanyl PVP | 2.00 |
| Butyloctyl Salicylate | 5.00 |
| Total | 100 |

### Example-7: Water-in-Silicone Mineral (Zinc) Emulsion

Method of preparation of Water-in-Silicone mineral (zinc) emulsion as per formulation of Table-7: Dimethicone 1cst and Dimethicone 2cst of Phase A were added to a main beaker and started mixing with a high lift mixing blade at high speed. Cetyl PEG/PPG-10/1 Dimethicone was then added and continued mixing at high speed. Polyglyceryl-4 Isostearate (and) Cetyl PEG/PPG-10/1 Dimethicone (and) Hexyl Laurate was then added to the mixture while stirring followed by Caprylic/Capric Triglyceride (and) Stearalkonium Hectorite (and) Propylene Carbonate. Butyloctyl Salicylate and Diisopropyl adipate of Phase B were added to a separate beaker and mixed until clear and uniform, Hydroxyethyl cellulose was then added and heated to a temperature of 80-85° C. until uniform and cooled down to a temperature of 70° C. It was then added to mixture of Phase A and continued mixing. DI water and Hydroxyethyl Cellulose of Phase C were mixed in a separate beaker and mixed at a temperature of 80-85° C. until dispersed well. Glycerin was then added to the mixture and continued mixing at a temperature of 85° C. until the Hydroxyethyl Cellulose was completely solubilized. Phenoxyethanol (and) Benzoic Acid (and) Dehydroacetic Acid and sodium chloride were then added to the mixture, continued mixing until sodium chloride was completely dissolved. The homogenized mixture of Phase C thus obtained was then slowly cooled down to room temperature and added to above mixture of Phase A with increased stirring to obtain a uniform mixture. Zinc Oxide (and) Triethoxycaprylylsilane of Phase D was then added to the mixture and continued stirring for 5 minutes until well homogenized. Switched the batch to a homogenizer and continued homogenizing for another 10 minutes.

TABLE 7
Water-in-Silicone mineral (zinc) emulsion.

| | |
|---|---|
| Ingredients | % w/w |

Phase A

| | |
|---|---|
| Dimethicone 1 cst | 5.00 |
| Dimethicone 2 cst | 10.00 |
| Cetyl PEG/PPG -10/1 Dimethicone | 0.50 |
| Polyglyceryl-4 Isostearate (and) Cetyl PEG/PPG-10/1 | 3.00 |
| Dimethicone (and) Hexyl Laurate | |
| Caprylic/Capric Triglyceride (and) Stearalkonium Hectorite | 3.50 |
| (and) Propylene Carbonate | |

Phase B

| | |
|---|---|
| Diisopropyl adipate | 1.50 |
| Butyloctyl Salicylate | 5.00 |
| Hydroxyethyl cellulose | 2.50 |

Phase C

| | |
|---|---|
| DI Water | 37.32 |
| Hydroxyethyl Cellulose | 0.20 |
| Glycerin | 2.00 |
| Benzyl o-toluate | 5.00 |
| Phenoxyethanol (and) Benzoic Acid (and) Dehydroacetic Acid | 1.00 |
| 1,2-Hexanediol | 0.50 |
| Sodium Chloride | 1.00 |

Phase D

| | |
|---|---|
| Zinc Oxide and Triethoxycaprylylsilane | 21.98 |
| Total | 100.00 |

### Example-8: Photostability of Avobenzone

10 minimal erythemal dose (MED) is a quantified dose of radiation that is known for causing skin damage. UV screening agents will eventually breakdown into smaller molecule after prolonged exposure to UV radiation. This is a common, expected phenomenon however, some UV screening agents will degrade faster within the time a person is exposed to sunlight. If the UV screening agent degrades, it loses the ability to absorb UV radiations and eventually results in a lower absorption profile, that translate into a lower SPF value.

If the absorption profile of a specimen, prepared from a UV screening agent-solvent combination, measured after a predefined exposure of 10 MED radiation, reads similar to that of its initial readings, then the UV screening agent is said to be stable in that particular combination of solvent. Any degradation of the UV screening agent during the exposure of 10 MED radiation will exhibit corresponding lower absorbance profile compared to its initial absorbance profile readings. Therefore, in such cases it can be concluded that the UV screening agent-solvent combination has failed to stabilize that UV screening agent.

Method of testing photostability: For each test formulations prepared according to Table-1 to Table-7, 1.80 mg/cm² of samples were deposited on PMMA (polymethyl methacrylate) plates and spread evenly using finger cots. Three plates were prepared for each product. The initial UV absorbance profile of each slide was measured with a UV-VIS spectrophotometer in the wavelength range of 290-400 nm. Four readings were taken for each slide. The test slides were then exposed to 10 MED radiation using a Solar Light LS1000R3 solar simulator. The slides were then equilibrated at room temperature for 15 minutes, and the UV absorbance profile post radiation was measured. The readings were then plotted to correlate initial absorbance behavior of the specimens to that of post radiation. Any degradation of the UV screening agent will result in a lower absorbance readings compared to that of the initial absorbance profile. Table-8 provides details photostability test results of avobenzone with various solvents measured using above method.

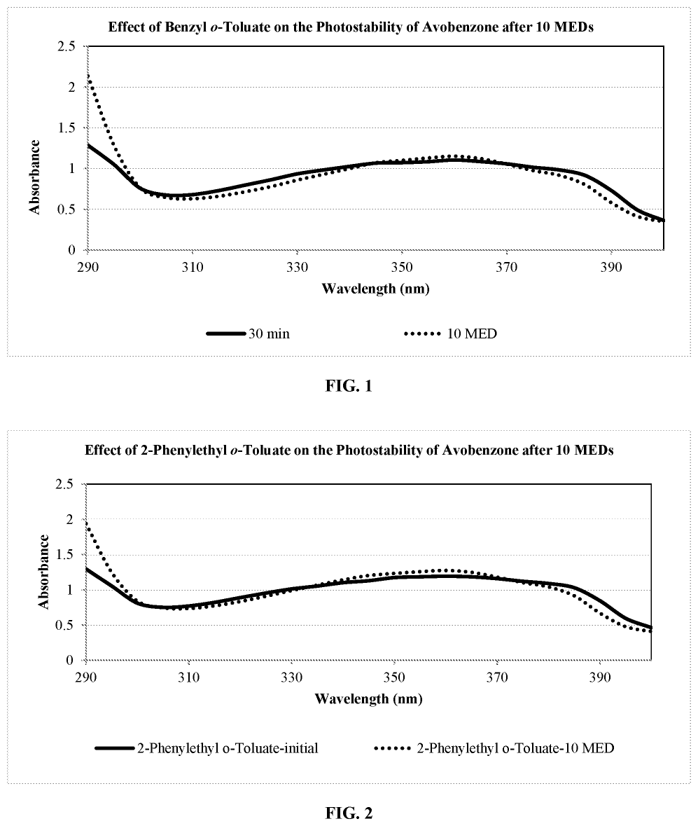

FIG. 1 to FIG. 6 illustrate graphs plotted based on UV absorbance profile measured before and after an exposure of 10 MED radiation on test specimens comprising combination of avobenzone and solvents selected from Table-8.

FIG. 1 shows graphs of UV absorbance profile measured before and after 10 MED radiation on test specimen comprising avobenzone and benzyl o-toluate. It is clear from FIG. 1 that the UV absorbance profile of avobenzone-benzyl o-toluate combination measured after 10 MED radiation is similar to that of initial absorbance profile readings. This indicates that avobenzone-benzyl o-toluate combination is stable.

FIG. 2 shows graphs of UV absorbance profile measured before and after 10 MED radiation on test specimen comprising avobenzone and 2-phenylethyl o-toluate. It is clear from FIG. 2 that the UV absorbance profile of avobenzone-2-phenylethyl o-toluate combination measured after 10 MED radiation is similar to that of initial absorbance profile readings. This indicates that avobenzone-2-phenylethyl o-toluate combination is stable.

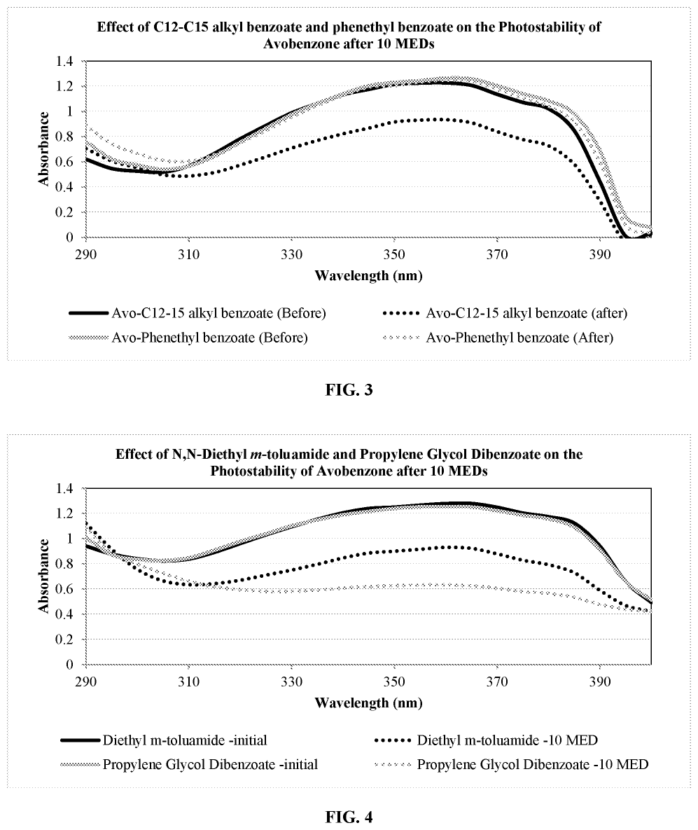

FIG. 3 shows graphs of UV absorbance profile measured before and after 10 MED radiation on separate test specimens prepared from combinations of (i) avobenzone and C12-C15 alkyl benzoate, and (ii) avobenzone and phenethyl benzoate. It is clear from FIG. 3 that the UV absorbance profile of combination (i) avobenzone and C12-C15 alkyl benzoate measured after 10 MED radiation are significantly low when compared to that of initial absorbance profile readings. This indicates that avobenzone is highly instable in combination with solvent C12-C15 alkyl benzoate. It can also be observed from FIG. 3 that the UV absorbance profile of combination (ii) avobenzone and phenethyl benzoate, measured after 10 MED radiation is similar to that of initial absorbance profile readings. This indicates that avobenzone-phenethyl benzoate combination is stable.

FIG. 4 shows graphs of UV absorbance profile measured before and after 10 MED radiation on separate test specimens prepared from combinations of (i) avobenzone and N,N-diethyl m-toluamide, and (ii) avobenzone and propylene glycol dibenzoate. It is clear from FIG. 4 that the UV absorbance profile of above samples measured after 10 MED radiation are significantly low when compared to that of initial absorbance profile readings. This indicates that avobenzone is highly instable in combination with solvents (i) N,N-diethyl m-toluamide, or (ii) propylene glycol dibenzoate.

FIG. 5 shows graphs of UV absorbance profile measured before and after 10 MED radiation on separate test specimens prepared from combinations of (i) avobenzone and 1,3-butylene glycol dibenzoate, and (ii) avobenzone and cyclohexyl benzoate. It is clear from FIG. 5 that the UV absorbance profile of above samples measured after 10 MED radiation are significantly low when compared to that of initial absorbance profile readings. This indicates that avobenzone is highly instable in combination with solvents (i) 1,3-butylene glycol dibenzoate, or (ii) cyclohexyl benzoate.

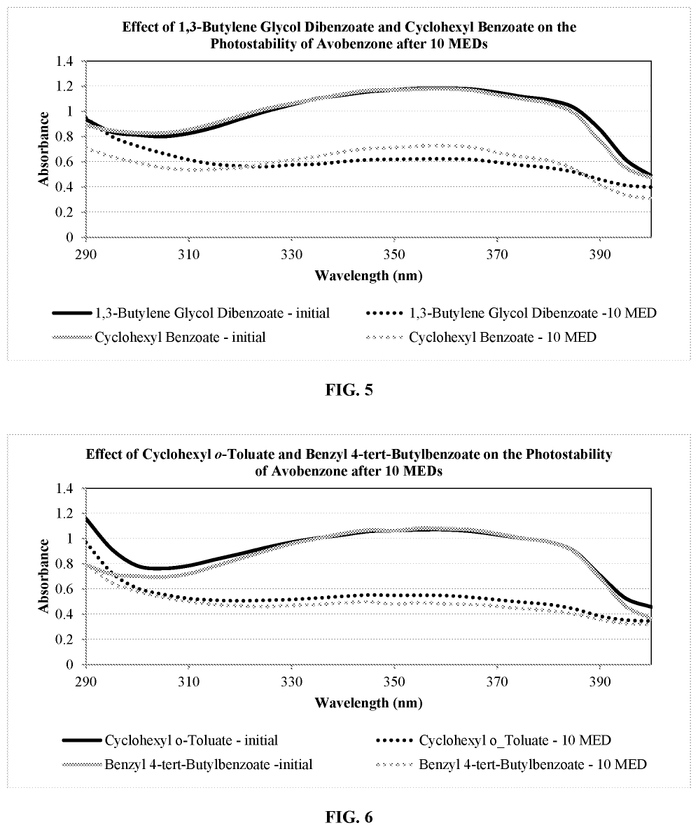

FIG. 6 shows graphs of UV absorbance profile measured before and after 10 MED radiation on separate test specimens prepared from combinations of (i) avobenzone and cyclohexyl o-toluate, and (ii) avobenzone and benzyl 4-tert-butylbenzoate. It is clear from FIG. 6 that the UV absorbance profile of above samples measured after 10 MED radiation are significantly low when compared to that of initial absorbance profile readings. This indicates that avobenzone is highly instable in combination with solvents (i) cyclohexyl o-toluate, or (ii) benzyl 4-tert-butylbenzoate.

### Example-9: Evaluation of Odor of Solvents

Samples of solvents according to Table-8 were tested to evaluate the odor.

Method of testing odor: A panel consisting of total 13 male and female members were formed for evaluation of odor of test samples. Fixed amount of each solvent according to Table-8 was placed in sealed containers and the panelist were asked to smell each sample. Panelists were then asked to rank the odor as good (acceptable) or bad (not acceptable). Good or bad was assigned against each sample based on the highest number of panelists that assign that score for each sample. Table-8 provides details of odor test results of samples according to above method.

TABLE 8
Test results for photostability and odor.

| | Odor of the | |
|---|---|---|
| Solvent name | composition | Stability of Avobenzone |
| Benzyl o-toluate | good | stable |
| 2-Phenylethyl o-toluate | good | stable |
| C12-C15 alkyl benzoate | good | not stable |
| Phenethyl benzoate | good | stable |
| N,N-Diethyl m-toluamide | good | not stable |
| Propylene glycol Dibenzoate | good | not stable |
| 1,3-Butylene glycol Dibenzoate | good | not stable |
| Cyclohexyl Benzoate | good | not stable |
| Cyclohexyl o-toluate | good | not stable |
| Benzyl 4-tert-Butylbenzoate | bad | not stable |
| Benzyl Benzoate | bad | stable |
| 3-Phenylpropyl Benzoate | bad | stable |
| 1-Phenylpropyl Benzoate | bad | not stable |
| o-Methylbenzyl Benzoate | bad | not stable |
| m-Methylbenzyl Benzoate | bad | not stable |
| 2-Cyclohexylethyl Benzoate | good | not stable |
| Benzyl m-toluate | bad | not stable |
| 1-Phenylethyl p-toluate | bad | not stable |
| Benzyl 4-Ethylbenzoate | bad | not stable |
| 2-Phenylethyl Cyclohexanoate | bad | not stable |
| 2-Phenylethyl Propionate | good | not stable |
| 2-Phenylethyl Hexanoate | bad | not stable |
| Benzyl Salicylate | good | not stable |

What is claimed is:

1. A sunscreen composition comprising:
(a) about 0.01 wt. % to about 20 wt. % of avobenzone;
(b) about 0.01 wt. % to about 20 wt. % of benzyl o-toluate; and,
(c) about 0.01 wt. % to about 95 wt. % of at least one additive.

2. The sunscreen composition according to claim 1, wherein the composition further comprises about 0.01 wt. % to about 25 wt. % of at least one UV screening agent, physical sun blocker and combinations thereof.

3. The sunscreen composition according to claim 2, wherein the UV screening agents and physical sun blockers are selected from the group consisting of octisalate, homosalate, octocrylene, benzopheneone-3, bis-ethylhexyloxyphenol methoxyphenyl triazine, butyloctyl salicylate p-minobenzoic acid and its derivatives, anthranilates, camphor derivatives, cinnamic derivatives, dibenzoyl methanes, β,β-diphenylacrylate derivatives, salicylic derivatives, triazine derivatives, benzimidazole compounds, bis-benzoazolyl derivatives, methylene bis-(hydroxyphenylbenzotriazole) compounds, oxyethylene (25 mol) p-aminobenzoate, 2-ethylhexyl p-dimethylaminobenzoate, ethyl N-oxypropylene p-aminobenzoate, glycerol p-aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammunium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, β-(2-oxoborn-3-ylidene)-tolyl-4-sulfonic acid and soluble salts thereof, 3-(4′-sulfo)benzylidenbornan-2-one and soluble salts thereof, 3-(4′methylbenzylidene)-d,l-camphor, 3-benzylidene-d,l-camphor, benzene 1,4-di(3-methylidene-10-camphosulfonic) acid, urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl)anilino]-1,3,5-triazine, N-(2 and 4)-[(2-oxoborn-3-yliden)methyl]benzyl]-acrylamide, 1,4-bisbenzimidazolyl-phenylen-3,3′,5,5′-tetrasulfonic acid, benzalmalonate-substituted polyorganosiloxanes, benzotriazole-substituted polyorganosiloxanes, dispersed 2,2′-methylene-bis-[6-(2H-benzotriazol-2-yl)-4-(1,1,3,3-tetramethylbutyl)phenol], solubilized 2,2′-methylene-bis-[6-(2H-benzotriazol-2-yl)-4-(methyl)phenol, cerium oxides, chromium oxides, cobalt oxides, iron oxides, red petrolatum, silicone-treated titanium dioxide, titanium dioxide, titanium dioxide (and) isopropyl titanium triisostearate, zirconium oxide, zinc oxide, zinc oxide (and) isopropyl titanium triisostearate, zinc oxide (and) triethoxycaprylylsilane, and acid, salts, esters, derivatives and combinations thereof.

4. The sunscreen composition according to claim 2, wherein the UV screening agents and physical sun blockers are selected from the group consisting of octisalate, homosalate, octocrylene, benzopheneone-3, bis-ethylhexyloxyphenol methoxyphenyl triazine, butyloctyl salicylate, titanium dioxide, titanium dioxide (and) isopropyl titanium triisostearate, zirconium oxide zinc oxide, zinc oxide (and) isopropyl titanium triisostearate, zinc oxide (and) triethoxycaprylylsilane, and combinations thereof.

5. The sunscreen composition according to claim 1, wherein the amount of avobenzone ranges from about 0.01 wt. % to about 10 wt. %.

6. The sunscreen composition according to claim 1,
wherein the benzyl o-toluate is used as a solubilizing and photostabilizing agent for avobenzone; and,
wherein the benzyl o-toluate is used as a solubilizing agent for bis-ethylhexyloxyphenol methoxyphenyl triazine.

7. The sunscreen composition according to claim 1, wherein the additive is selected from the group consisting of emulsifiers, humectants, emollients, preservatives, buffering agents and/or pH adjusters, rheology modifiers, liquid carriers, film forming and/or water proofing agent, chelating agents, siloxanes, emulsion stabilizers, fatty acid esters, solvents, propellants, surfactants, skin-feel additives, anti-aging agents, exfoliating agents, defoamers, dyes, fragrances, waxes, conditioners, lubricants, antibiotics, viscosity synergists, clarity synergists, antioxidants, and combinations thereof.

8. The sunscreen composition according to claim 7, wherein the emulsifiers are selected from the group consisting of C16-C18 alkyl alcohol ethoxylate, glyceryl stearate, glyceryl stearate SE, glyceryl Stearate (and) PEG-100 stearate, dimethicone (and) dimethicone/PEG-10/15 crosspolymer, cetyl PEG/PPG-10/1 Dimethicone, polyglyceryl-4 isostearate (and) cetyl PEG/PPG-10/1 dimethicone (and) hexyl laurate, lauryl PEG-9 polydimethylsiloxyethyl dimethicone, glyceryl laurate, glyceryl dilaurate, glycol distearate, sorbitan trioleate, glycol stearate, sorbitan sesquioleate, lecithin, sorbitan oleate, sorbitan stearate, sorbitan isostearate, oleth-2, steareth-2, calcium stearoyl lactylate, ceteth-2, PEG-4 dilaurate, sorbitan stearate (and) sucrose cocoate, methyl glucose sesquistearate, sucrose stearate, PEG-10 sunflower glycerides, sodium stearoyl lactylate, sorbitan laurate, PEG-40 sorbitan peroleate, lecithin, laureth-4, PEG-7 glyceryl cocoate, PEG-20 almond glycerides, linoleamide DEA, PEG-25 hydrogenated castor oil, cetearyl glucoside, polysorbate 85, glyceryl stearate (and) PEG-100 stearate, stearamide MEA, oleth-10/polyoxyl 10 oleyl ether NF, oleth-10, oleth-20, ceteth-10, PEG-8 lauratc, cocamide MEA, polysorbate 60, PEG-60 almond glycerides, isosteareth-20, lauramide DEA, PEG-30 dipolyhydroxystearate, polysorbate 80, PEG-20 methyl glucose sesquistearate, oleth-20, steareth-21, isoceteth-20, polysorbate 20, laureth-23, PEG-100 stearate, PEG-80 sorbitan laurate, potassium cetyl phosphate, cetearyl alcohol, diacetyl phosphate, ceteth-10 phosphate, and combinations thereof.

9. The sunscreen composition according to claim 7, wherein the humectants are selected from the group consisting of glycerin, polyethylene glycol, propylene glycol, ethylene glycol, diethylene glycol, propylene glycol, methyl alcohol, ethyl alcohol, propyl alcohol, isopropyl alcohol, glycerol, sorbitol, and combinations thereof.

10. The sunscreen composition according to claim 7, wherein the emollients are selected from the group consisting of cetyl Alcohol, isocetyl alcohol, isodecyl neopentanoate, isostearyl neopentanoate and isocetyl stearyl stearate, diisopropyl adipate, lauryl lactate, octyl palmitate, C12-C15 alkyl lactate, dicaprylyl carbonate, isodecyl salicylate, glycerin, glycerin (and) glyceryl acrylate/acrylic acid copolymer (and) PVM/MA Copolymer, octyl palmitate, diisopropyl adipate, and combinations thereof.

11. The sunscreen composition according to claim 7, wherein the preservatives are selected from the group consisting of propylene glycol (and) diazolidinyl urea (and) methylparaben (and) propylparaben, 1,2-Hexanediol, methyl 4-hydroxybenzoate, phenoxyethanol (and) benzoic acid (and) dehydroacetic acid, lower alkyl esters of para-hydroxybenzoates (parabens) selected from methylparaben, propylparaben, isobutylparaben, phenoxyethanol (and) methylparaben (and) ethylparaben (and) propylparaben; benzyl alcohol, phenoxyethanol, benzoic acid, caprylyl glycol (and) phenoxyethanol and combinations thereof.

12. The sunscreen composition according to claim 7, wherein the buffering agents and/or pH adjusters are selected from the group consisting of triethanolamine, sodium diacetate, sodium acetate, sodium succinate, disodium succinate, sodium citrate, sodium formate, sodium hydroxide, sodium or ammonium salts, and combinations thereof.

13. The sunscreen composition according to claim 7, wherein the rheology modifiers are selected from the group consisting of polyacrylates, acrylate copolymers, acrylic acid/vinylpyrrolidone cross polymer, acrylates (and) C10-C30 alkyl acrylate cross polymer, methyl cellulose, ethyl cellulose, hydroxyethyl cellulose, hydroxypropyl methyl cellulose, stearalkonium hectorite (and) caprylic/capric triglyceride (and) propylene carbonate, and combinations thereof.

14. The sunscreen composition according to claim 7, wherein the liquid carriers are selected from the group consisting of alcohols, water, natural oils, and combinations thereof.

15. The sunscreen composition according to claim 8, wherein the chelating agents are selected from the group consisting of tetrasodium EDTA, disodium pyrophosphate, dipotassium pyrophosphate, tetrapotassium pyrophosphates, tetrasodium pyrophosphates, disodium ethane-1-hydroxy-1,1-diphosphonate (EHDP), methanediphosphonic acid, 2-phosphonobutane-1,2,4-tricarboxylic acid, citric acid, sodium citrate, potassium citrate, tartaric acid, disodium calcium ethylene diamine tetraacetic acid, disodium ethylene diamine tetraacetate, disodium ethylene diamine tetraacetic acid, gluconic acid, methyl vinyl ether and maleic anhydride (PVM/MA) copolymers, polyacrylic acid, polyitaconic acid and combinations thereof.

16. The sunscreen composition according to claim 7, wherein the film forming and/or water proofing agents are selected from the group consisting of vinyl acetate (and) butyl maleate (and) isobornyl acrylate copolymer, vinylpyrrolidone (and) eicosene copolymer, vinylpyrrolidone (and) acrylates (and) lauryl methacrylate copolymer; vinyl pyrrolidone (and) 1-triacontane copolymer, vinylpyrrolidone (and) hexadecene copolymer, poly (butylated vinylpyrrolidone), vinylpyrrolidone (and) hexadecene copolymer, acrylates (and) C12-C22 alkylmethacrylate copolymer, Tricontanyl polyvinylpyrrolidone, hydroxyethyl cellulose, and combinations thereof.

17. The sunscreen composition according to claim 7, wherein the siloxanes are selected from the group consisting of dimethylpolysiloxane, cyclopentasiloxane, cyclopentasiloxane (and) dimethylpolysiloxane (and) vinyltrimethylsiloxysilicate cross polymer, polyalkyl siloxanes, polyaryl siloxanes, phenyl trimethicone, poly alkyl aryl siloxane, an organic functional group modified poly-organic siloxane and combinations thereof.

18. The sunscreen composition according to claim 1, wherein the sunscreen composition is in the form of a lotion, an oil-in-water emulsion, a water-in-oil emulsion, a spray, a water-in-silicone emulsion, an oily solution, a lipid fusion, a hydro-alcoholic gel, an anhydrous gel, an aqueous gel, an alcoholic solutions, or a hydro-alcoholic solution.

19. A method for solubilizing and photostabilizing avobenzone in a sunscreen composition comprising avobenzone, the method comprising adding about 0.01 wt. % to about 20 wt. % of benzyl o-toluate to the composition to stabilize the avobenzone against photodegradation and to effectively solubilizes avobenzone.

20. The method according to claim 19, wherein the sunscreen composition further comprises at least one UV screening agent or physical sun blocker and combinations thereof.

21. A method for protecting the skin against UV radiation comprising, applying to the skin an effective amount of a sunscreen composition of claim 1.

22. The method according to claim 21, wherein the skin is human skin or animal skin.
